(19) United States
(12) Patent Application Publication
(10) Pub. No.: US 2025/0255900 A1
(43) Pub. Date: Aug. 14, 2025

(54) COMPOSITIONS FOR IMPROVING HEALTH

(71) Applicant: Shaman Naturals, LLC, Casper, WY (US)

(72) Inventor: Maria Crisler, Casper, WY (US)

(21) Appl. No.: 19/116,433

(22) PCT Filed: Oct. 2, 2023
(86) PCT No.: PCT/US2023/075673

§ 371 Date: Mar. 28, 2025

Related U.S. Application Data
(60) Provisional application No. 63/412,030, filed on Sep. 30, 2022.

(51) Int. Cl. A61K 33/30; A61K 9/00; A61K 31/00; A61K 31/045; A61K 31/198; A61K 31/352; A61K 31/375; A61K 38/06; A61K 45/06; A61K 47/24; A61K 47/44

(52) U.S. Cl. CPC A61K 33/30; A61K 9/0014; A61K 9/006; A61K 31/045; A61K 31/198; A61K 31/352; A61K 31/375; A61K 31/658; A61K 38/063; A61K 45/06; A61K 47/24; A61K 47/44

(57) ABSTRACT

Disclosed herein are compositions comprising a long chain alcohol comprising octacosanol, hexacosanol, and/or tricontanol in an amount of at least 50% by weight based on total weight of the long chain alcohol; a cannabinoid; and a carrier. Also disclosed herein are compositions comprising a long chain alcohol; a cannabidiol; a vitamin; an ionophore; an amino acid; zinc; glutathione; and a carrier. Also disclosed are topical compositions and tinctures comprising any of the compositions disclosed herein. Also disclosed are methods of treating a disorder comprising administered any of the compositions disclosed herein.

### CROSS-REFERENCE TO RELATED APPLICATION

This application claims priority to U.S. Provisional Application No. 63/412,030 entitled “COMPOSITIONS FOR IMPROVING HEALTH,” filed on Sep. 30, 2022.

### TECHNICAL FIELD

The present disclosure relates to compositions comprising cannabinoids for use in improving health.

### BACKGROUND

Numerous diseases and disorders are linked to oxidative stress, including, for example, neurodegenerative disease, inflammation, metabolic disorders, cancer, and atherosclerosis. Oxidative stress refers to an imbalance between the amount of reactive oxygen species present within a cell and the cell's ability to neutralize the reactive oxygen species.

It would be advantageous to develop compositions that aid in the cell's ability to neutralize reactive oxygen species in order to improve health.

### SUMMARY

The present disclosure is directed to a composition comprising a long chain alcohol comprising octacosanol, hexacosanol, and/or tricontanol in an amount of at least 50% by weight based on total weight of the long chain alcohol; a cannabinoid; and a carrier.

The disclosure is further directed to a composition comprising a long chain alcohol; a cannabidiol; a vitamin; an ionophore; an amino acid; a zinc; glutathione; and a carrier.

The disclosure is further directed to a topical composition comprising any of the compositions disclosed herein.

The disclosure is further directed to a tincture comprising any of the compositions disclosed herein.

The disclosure is further directed to methods comprising administering to a subject any of the compositions disclosed herein.

### DETAILED DESCRIPTION

For purposes of the following detailed description, it is to be understood that the invention may assume various alternative variations and step sequences, except where expressly specified to the contrary. Moreover, other than in any operating examples, or where otherwise indicated, all numbers such as those expressing values, amounts, percentages, ranges, subranges, and fractions may be read as if prefaced by the word “about,” even if the term does not expressly appear. Accordingly, unless indicated to the contrary, the numerical parameters set forth in the following specification and attached claims are approximations that may vary depending upon the desired results to be obtained. At the very least, and not as an attempt to limit the application of the doctrine of equivalents to the scope of the claims, each numerical parameter should at least be construed in light of the number of reported significant digits and by applying ordinary rounding techniques. Where a closed or open-ended numerical range is described herein, all numbers, values, amounts, percentages, subranges, and fractions, within or encompassed by the numerical range are to be considered as being specifically included in and belonging to the original disclosure of this application as if these numbers, values, amounts, percentages, subranges, and fractions have been explicitly written out in their entirety.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard variation found in their respective testing measurements.

As used herein, unless indicated otherwise, a plural term can encompass its singular counterpart and vice versa. For example, although reference is made herein to “a” cannabinoid, “a” phospholipid, or “an” amino acid, a combination (i.e., a plurality) of these components can be used. In addition, in this application, the use of “or” means “and/or” unless specifically stated otherwise, even though “and/or” may be explicitly used in certain instances.

As used herein, “including,” “containing,” and like terms are understood in the context of this application to be synonymous with “comprising” and are therefore open-ended and do not exclude the presence of additional undescribed and/or unrecited elements, materials, ingredients, and/or method steps.

As used herein, “consisting of” is understood in the context of this application to exclude the presence of any unspecified element, ingredient, and/or method step.

As used herein, “consisting essentially of” is understood in the context of this application to include the specified elements, materials, ingredients, and/or method steps “and those that do not materially affect the basic and novel characteristic(s)” of what is being described.

As used herein, the terms “patient,” “subject,” “individual,” and the like are used interchangeably herein and mean animals amenable to the methods of treatment described herein, including mammals, including humans, a canine, a feline, a bovine, an equine, a porcine, a primate, and/or a rodent.

As used herein, “administering” or “administration” of an amount (e.g., a dose) of a composition may be done by the subject himself/herself or another subject (e.g., a medical professional, a caretaker, or a family member). The composition may be provided to the subject or the administrator for the subject along with instructions for the administration of the composition (e.g., written instructions on the label of a container containing the composition). The composition may be introduced or delivered by any route in order to perform the intended function, including but not limited to topically or orally, such as sublingually.

As used herein, “disorder,” “disease,” and “illness” are used interchangeably and refer to a condition in a subject that negatively impacts the health of the subject.

As used herein, “treat,” “treatment,” or “treating” means treatment of a disease or disorder, as defined herein, in a subject, including: (1) inhibiting a disease or disorder; (2) arresting the development of a disease or disorder; (3) slowing progression of the disease or disorder; and/or (4) inhibiting, relieving, or slowing progression of one or more symptoms of the disease or disorder. A disease, disorder, or illness is “treated” if the subject experiences a reduction in the severity and/or frequency of the disease, disorder, and/or illness or a symptom associated therewith.

As used herein, a “therapeutically effective amount” refers to an amount required to (1) maintain homeostasis in a patient and/or (2) ameliorate the symptoms of a disease in a treated patient suffering the disease relative to an untreated patient suffering the disease. The effective amount of active compound(s) varies depending upon the manner of administration, as well as the age, body weight, and general health of the subject.

As used herein, “measuring” or “measurement” or alternatively “detecting” or “detection” means assessing the presence, absence, quantity, or amount (which can be any effective amount) of either a given substance within a clinic or subject-driven sample, including the derivation of qualitative or quantitative levels of such substance, or otherwise evaluating the values or categorization of a subject's clinical parameters.

As used herein, “sample” or “biological sample” as used herein refers to a biological material isolated from an individual. The biological sample may contain any biological material suitable for detecting the desired biomarkers and may comprise cellular and/or non-cellular material obtained from the individual.

As used herein, “composition” refers to a solution, dispersion, or solid, such as a powder.

As used herein, “carrier” encompasses carriers, excipients, and diluents, meaning a material, composition, or vehicle, such as a liquid or solid filler, diluent, excipient, solvent, or encapsulating material involved in carrying or transporting a pharmaceutical, cosmetic, or other agent across a phospholipid bilayer, e.g., a cellular membrane or organelle membrane.

As used herein, a “derivative” is a chemical that is related structurally to a first chemical and theoretically derived from it; a compound that is formed from a similar first compound or a compound that can arise from another first compound, if one atom of the first compound is replaced with another atom or group of atoms; a compound derived or obtained from a parent compound containing essential elements of the parent compound; or a chemical compound that may be produced from a first compound of similar structure in one or more steps.

As used herein, “precursor” refers to a compound that may be used in a chemical reaction to produce the desired compound. For example, cannabidiolic acid (“CBDA”) may be modified, partially digested, or otherwise acted upon by enzymes in the body to produce cannabidiol (CBD) or cannabigerol (CBG), which may be the active form of cannabidiol in composition.

As used herein, “prodrug” refers to a pharmacologically inactive compound or molecule that, upon administration to a subject, is metabolized into a pharmacologically active compound or molecule. For purposes of this disclosure, “precursor” may encompass “prodrug.”

Disclosed herein are compositions comprising, consisting essentially of, or consisting of a long chain alcohol comprising octacosanol, hexacosanol, and/or triacontanol in an amount of at least 50% by weight based on total weight of the long chain alcohol; a cannabinoid; and a carrier.

Also disclosed herein are compositions comprising, or consisting essentially of, or consisting of: a long chain alcohol; a cannabidiol; a vitamin; an ionophore; an amino acid; zinc; glutathione; and a carrier.

As disclosed herein, the composition may comprise, consist essentially of, or consist of a long chain alcohol. The long chain alcohol may comprise at least one hydroxy functional group on a saturated, unsaturated, or polyunsaturated carbon chain. The carbon chain may comprise at least 20 carbons, such as at least 22 carbons, such as at least 24 carbons. The carbon chain may comprise no more than 40 carbons, such as no more than 36 carbons, such as no more than 34 carbons. The carbon chain may comprise 20 to 40 carbons, such as 20 to 36 carbons, such as 22 to 36 carbons, such as 22 to 34 carbons, such as 24 to 34 carbons. The at least one hydroxy functional group may be a terminal or pendant group.

The long chain alcohol may comprise, consist essentially of, or consist of octacosanol, hexacosanol, triacontanol, docosanol, dotriacontanol, tetracosanol, or combinations thereof.

The long chain alcohol may comprise octacosanol, hexacosanol and/or triacontanol in an amount of at least 50% by weight based on total weight of the long chain alcohol, such as at least 60% by weight, such as at least 70% by weight, such as at least 80% by weight, such as at least 90% by weight, such as 100% by weight. The long chain alcohol may comprise octacosanol, hexacosanol, and/or triacontanol in an amount of no more than 95% by weight based on total weight of the long chain alcohol, such as no more than 90% by weight, such as no more than 85% by weight, such as no more than 80% by weight. The long chain alcohol may comprise octacosanol, hexacosanol and/or triacontanol in an amount of 50% to 100% by weight based on total weight of the long chain alcohol, such as 60% to 100% by weight, such as 60% to 95% by weight, such as 60% to 90% by weight, such as 70% to 100% by weight, such as 70% to 95% by weight, such as 70% to 90% by weight, such as 80% to 100% by weight, such as 80% to 95% by weight, such as 80% to 90% by weight.

The long chain alcohol may comprise octacosanol in an amount of at least 40% by weight based on total weight of the alcohol, such as at least 45% by weight, such as at least 50% by weight, such as at least 55% by weight, such as at least 60% by weight. The long chain alcohol may comprise octacosanol in an amount of no more than 90% by weight based on total weight of the long chain alcohol, such as no more than 85% by weight, such as no more than 80% by weight, such as no more than 75% by weight, such as no more than 70% by weight. The long chain alcohol may comprise octacosanol in an amount of 40% to 90% by weight based on total weight of the long chain alcohol, such as 45% to 85% by weight, such as 50% to 80% by weight, such as 55% to 75% by weight, such as 60% to 70% by weight.

The long chain alcohol may comprise hexacosanol in an amount of at least 5% by weight based on total weight of the long chain alcohol, such as at least 7% by weight, such as at least 10% by weight. The long chain alcohol may comprise hexacosanol in an amount of no more than 50% by weight based on total weight of the long chain alcohol, such as no more than 40% by weight, such as no more than 30% by weight, such as no more than 20% by weight, such as no more than 15% by weight. The long chain alcohol may comprise hexacosanol in an amount of 5% to 50% by weight based on total weight of the long chain alcohol, such as 5% to 40% by weight, such as 5% to 30% by weight, such as 5% to 20% by weight, such as 5% to 15% by weight, such as 7% to 50% by weight, such as 7% to 40% by weight, such as 7% to 30% by weight, such as 7% to 20% by weight, such as 7% to 15% by weight, such as 10% to 50% by weight, such as 10% to 40% by weight, such as 10% to 30% by weight, such as 10% to 20% by weight, such as 10% to 15% by weight.

The long chain alcohol may comprise triacontanol in an amount of at least 5% by weight based on total weight of the long chain alcohol, such as at least 7% by weight, such as at least 10% by weight. The long chain alcohol may comprise triacontanol in an amount of no more than 50% by weight based on total weight of the long chain alcohol, such as no more than 40% by weight, such as no more than 30% by weight, such as no more than 20% by weight. The long chain alcohol may comprise triacontanol in an amount of 5% to 50% by weight based on total weight of the long chain alcohol, such as 5% to 40% by weight, such as 5% to 30% by weight, such as 5% to 20% by weight, such as 7% to 50% by weight, such as 7% to 40% by weight, such as 7% to 30% by weight, such as 7% to 20% by weight, such as 10% to 50% by weight, such as 10% to 40% by weight, such as 10% to 30% by weight, such as 10% to 20% by weight.

The long chain alcohol may comprise a ratio of hexacosanol to octacosanol of at least 1:19, such as at least 1:9, such as at least 3:17. The long chain alcohol may comprise a ratio of hexacosanol to octacosanol of no more than 2:3, such as no more than 3:7, such as no more than 1:4. The long chain alcohol may comprise a ratio of hexacosanol to octacosanol of 1:19 to 2:3, such as 1:9 to 3:7, such as 3:17 to 1:4.

The long chain alcohol may comprise a ratio of hexacosanol to triacontanol of at least 1:4, such as at least 3:7, such as at least 2:3, such as 1:1. The long chain alcohol may comprise a ratio of hexacosanol to triacontanol of no more than 4:1, such as no more than 7:3, such as no more than 3:2. The long chain alcohol may comprise a ratio of hexacosanol to triacontanol of 1:4 to 4:1, such as 1:4 to 1:1, such as 3:7 to 7:3, such as 3:7 to 1:1, such as 2:3 to 3:2, such as 2:3 to 1:1.

The long chain alcohol may comprise a ratio of triacontanol to octacosanol of at least 1:19, such as at least 1:9, such as at least 3:17. The long chain alcohol may comprise a ratio of triacontanol to octacosanol of no more than 2:3, such as no more than 3:7, such as no more than 1:4. The long chain alcohol may comprise a ratio of triacontanol to octacosanol of 1:19 to 2:3, such as 1:9 to 3:7, such as 3:17 to 1:4.

The long chain alcohol may be in a powder form at ambient conditions. As used herein, “ambient conditions” refers to room temperature (e.g., 23° C.) and humidity conditions, e.g., at 10° C. to 40° C. and 5% to 80% relative humidity.

The long chain alcohol may have a bulk density of at least 0.3 g/mL as measured by USP 616, such as at least 0.35 g/mL, such as at least 0.4 g/mL. The long chain alcohol may have a bulk density of no more than 0.6 g/mL as measured by USP 616, such as no more than 0.55 g/mL, such as no more than 0.5 g/mL. The long chain alcohol may have a bulk density of 0.3 g/mL to 0.6 g/mL as measured by USP 616, such as 0.35 g/mL to 0.55 g/mL, such as 0.4 g/mL to 0.5 g/mL.

The long chain alcohol may have a tapped density of at least 0.2 g/mL as measured by USP 616, such as at least 0.25 g/mL, such as at least 0.3 g/mL. The long chain alcohol may have a tapped density of no more than 0.8 g/mL as measured by USP 616, such as no more than 0.75 g/mL, such as no more than 0.7 g/mL. The long chain alcohol may have a tapped density of 0.2 g/mL to 0.8 g/mL as measured by USP 616, such as 0.25 g/mL to 0.75 g/mL, such as 0.3 g/mL to 0.7 g/mL.

The long chain alcohol may be derived from any source, including but not limited to sugar cane, beeswax, cereal grains, grasses, leaves, fruits, buds, seeds, or combinations thereof.

The long chain alcohol may comprise nanoparticles, such as a nanoemulsion comprising nanoparticles. The nanoparticles may comprise a particle size of no more than 100 nm, such as a positive particle size up to 100 nm, such as up to 10 nm, such as up to 20 nm, such as up to 30 nm, such as up to 40 nm, such as up to 50 nm, such as up to 60 nm, such as up to 70 nm, such as up to 80 nm, such as up to 90 nm. The nanoparticles may comprise a long chain alcohol and a stabilizer. The stabilizer may comprise, consist essentially of, or consist of polyethylene glycol ester, tocopheryl ester, tocopheryl polyethylene glycol ester, tocopheryl polyethylene glycol (1000) succinate (“TPGS”), or combinations thereof.

The concentration of the long chain alcohol will vary based upon, for example, the target organ or tissue, the disease indication being treated, and/or the projected length of treatment.

The composition may comprise the long chain alcohol in an amount of at least 0.25% by weight based on total weight of the composition, such as at least 0.50% by weight, such as at least 0.75% by weight, such as at least 1.0% by weight. The composition may comprise the long chain alcohol in an amount of no more than 5.0% by weight based on total weight of the composition, such as no more than 4.0% by weight, such as no more than 3.0% by weight, such as no more than 2.0% by weight. The composition may comprise the long chain alcohol in an amount of 0.25% to 5.0% by weight based on total weight of the composition, such as 0.50% to 4.0% by weight, such as 0.75% to 3.0% by weight, such as 1.0% to 3.0% by weight, such as 1.0% to 2.0% by weight.

The compositions disclosed herein may comprise, consist essentially of, or consist of a cannabinoid. As used herein, “cannabinoid” refers to a chemical compound that shows direct or indirect activity at a cannabinoid receptor, e.g., CB₁ and/or CB₂. A cannabinoid may be a phytocannabinoid, an endocannabinoid, or a synthetic cannabinoid. The term “phytocannabinoid” refers to a cannabinoid that occurs in a plant species or is derived from cannabinoids occurring in a plant species. The term “synthetic cannabinoid” refers to a cannabinoid that has been artificially manufactured. The term “endocannabinoid” refers to a cannabinoid that occurs in an animal species or is derived from cannabinoids occurring in an animal species. Examples of phytocannabinoids that may be used in the present disclosure include, but are not limited to, cannabigerolic acid (CBGA), cannabigerol (CBG), cannabigerol monomethylether (CBGM), cannabigerovarin (CBGV), cannabichromene (CBC), cannabidiol monomethylether (CBDM), cannabidiol-C4 (CBD-C4), cannabidivarin (CBDV), cannabidiorcol (CBD-C1), delta-9-tetrahydrocannabinol (W-THC), delta-9-tetrahydrocannabinolic acid (THCA-A), delta-9-tetrahydrocannabionolic acid B (THCA-B), delta-9-tetrahydrocannabinolic acid-C4 (THCA-C4), delta-9-tetrahydrocannabinol-C4, delta-9-tetrahydrocannabivarin (THCV), delta-9-tetrahydrocannabiorcol (THC-C1), delta-7-cis-iso tetrahydrocannabivarin, delta-8-tetrahydrocannabinol (As-THC), cannabicyclol (CBL), cannabicyclovarin (CBLV), cannabielsoin (CBE), cannabinol (CBN), cannabinol methylether (CBNM), cannabinol-C4 (CBN-C4), cannabivarin (CBV), cannabinol-C2 (CBN-C2), cannabiorcol (CBN-C1), cannabinodiol (CBND), cannabinodivarin (CBVD), cannabitriol (CBT), 10-ethoxy-9-hydroxy-delta-6a-tetrahydrocannabinol, cannabitriolvarin (CBTV), ethoxy-cannabitriolvarin (CBTVE), dehydrocannabifuran (DCBF), cannabifuran (CBF), cannabichromanon (CBCN), cannabicitran (CBT), 10-oxo-delta-6a-tetrahydrocannabinol (OTHC), delta-9-cis-tetrahydrocannabinol (cis-THC), 3,4,5,6-tetrahydro-7-hydroxy-alpha-alpha-2-trimethyl-9-n-propyl-2, 6-methano-2H-1-benzoxocin-5-methanol (OH-iso-HHCV), cannabiripsol (CBR), trihydroxy-delta-9-tetrahydrocannabinol (triOH-THC), cannabinol propyl variant (CBNV), derivatives thereof, and combinations thereof.

Examples of synthetic cannabinoids include, but are not limited to, naphtholyindoles, naphthylmethylindoles, naphthoylpyrroles, naphthyl methyl indenes, phenylacetylindoles, cyclohexylphenols, tetramethylcyclopropylindoles, adamantoylindoles, indazole carboxamides, quinolinyl esters, and combinations thereof.

As disclosed herein, the cannabinoid may comprise, consist essentially of, or consist of a cannabidiol. As used herein, the term “cannabidiol” refers to a phytocannabinoid that does not have a psychoactive effect. The term “cannabidiol” may refer to one or more of the compounds Δ⁵-cannabidiol (2-(6-isopropenyl-3-methyl-5-cyclohexen-1-yl)-5-pentyl-1,3-benzenediol); Δ⁴-cannabidiol (2-(6-isopropenyl-3-methyl-4-cyclohexen-1-yl)-5-pentyl-1,3-benzenediol); Δ³-cannabidiol (2-(6-isopropenyl-3-methyl-5-cyclohexen-1-yl)-5-pentyl-1,3-benzenediol); Δ³′7-cannabidiol (2-(6-isopropenyl-3-methyl-5-cyclohexen-1-yl)-5-pentyl-1,3-benzenediol); Δ²-cannabidiol (2-(6-isopropenyl-3-methyl-5-cyclohexen-1-yl)-5-pentyl-1,3-benzenediol); Δ¹-cannabidiol (2-(6-isopropenyl-3-methyl-5-cyclohexen-1-yl)-5-pentyl-1,3-benzenediol); Δ⁶-cannabidiol (2-(6-isopropenyl-3-methyl-5-cyclohexen-1-yl)-5-pentyl-1,3-benzenediol); derivatives thereof; and precursors thereof, including cannabidiolic acid (“CBDA”).

A cannabinoid may be in an acid form or a non-acid form, the latter also being referred to as the decarboxylated form. Within the context of the present disclosure, where reference is made to a particular cannabinoid, reference thereof encompasses the acid form, the non-acid form, or combinations thereof.

The cannabinoid may be extracted from a cannabis plant. For example, the cannabinoid may be extracted from the roots, the stems, the stalks, the leaves, the flowers, and/or the seeds of a cannabis plant. Examples of species from which the cannabinoid may be extracted include but are not limited to C. sativa, C. indica, and C. ruderalis. In examples, the cannabinoid is extracted from hemp.

The cannabidiol may be present in the composition as a precursor, such as a prodrug, such as CBDA.

The composition may comprise cannabinoids in an amount of at least 0.1% by weight based on total weight of the composition, such as at least 0.5% by weight, such as at least 1.0% by weight. The composition may comprise cannabinoids in an amount of no more than 5.0% by weight based on total weight of the composition, such as no more than 4.0% by weight, such as no more than 3.0% by weight, such as no more than 2.0% by weight. The composition may comprise cannabinoids in an amount of 0.1% to 5.0% by weight based on total weight of the composition, such as 0.5% to 4.0% by weight, such as 1.0% to 3.0% by weight, such as 1.0% to 2.0% by weight.

The cannabinoid may comprise any combination of the cannabinoids disclosed herein. Optionally, the cannabinoid may comprise cannabidiol in an amount of over 50% by weight based on total weight of the cannabinoid, such as at least 60% by weight, such as at least 70% by weight, such as at least 80% by weight, such as at least 90% by weight, such as at least 95% by weight, such as at least 99% by weight, such as at least 99.5% by weight, such as at least 99.9% by weight, such as 100% by weight.

The cannabinoid may comprise a cannabinoid in addition to the cannabidiol in an amount of less than 50% by weight based on total weight of the cannabinoid, such as no more than 40% by weight, such as no more than 30% by weight, such as no more than 20% by weight, such as no more than 10% by weight, such as no more than 5% by weight, such as no more than 1% by weight, such as no more than 0.5% by weight, such as no more than 0.25% by weight, such as no more than 0.1% by weight.

The composition may comprise less than 0.1% by weight of a cannabinoid in addition to the cannabidiol based on total weight of the composition, such as less than 0.05% by weight, such as less than 0.03% by weight. The composition may comprise at least 0.005% by weight of a cannabinoid in addition to the cannabidiol based on total weight of the composition, such as at least 0.008% by weight, such as at least 0.01% by weight. The composition may comprise from 0.005% to less than 0.1% by weight of a cannabinoid in addition to the cannabidiol based on total weight of the composition, such as 0.008% to 0.05% by weight, such as 0.01% to 0.03% by weight.

In examples, the cannabinoid may be substantially free, essentially free, or completely free of a cannabinoid other than cannabidiol.

The composition further comprises, consists essentially of, or consists of a carrier.

The carrier may comprise, consist essentially of, or consist of a phospholipid and/or water. Suitable phospholipids include but are not limited to polyoxyethylene, sorbitan monolaurate, soritan monooleate, polyethylene glycol-hydrogenated castor oil (e.g., cremophor and cremophor RH), egg phospholipid, a soy phospholipid, lecithin, such as sunflower lecithin, olive oil, grapeseed oil, tea tree oil, almond oil, avocado oil, sesame oil, evening primrose oil, sunflower oil, kukui nut oil, jojoba oil, walnut oil, peanut oil, pecan oil, macadamia nut oil, coconut oil, and combinations thereof. To the extent that the carrier may act as an ionophore it is not included as an ionophore for purposes of this disclosure.

The composition may comprise the carrier in an amount of at least 50% by weight based on total weight of the composition, such as at least 60% by weight, such as at least 70% by weight, such as at least 80% by weight. The composition may comprise the carrier in an amount of no more than 95% by weight based on total weight of the composition, such as no more than 94% by weight, such as no more than 93% by weight, such as no more than 90% by weight. The composition may comprise the carrier in an amount of 50% to 95% by weight based on total weight of the composition, such as 60% to 94% by weight, such as 70% to 93% by weight, such as 80% to 90% by weight.

The composition may comprise the phospholipid in an amount of at least 30% by weight based on total weight of the composition, such as at least 35% by weight, such as at least 40% by weight. The composition may comprise the phospholipid in an amount of no more than 70% by weight based on total weight of the composition, such as no more than 60% by weight, such as no more than 50% by weight. The composition may comprise the phospholipid in an amount of 30% to 70% by weight based on total weight of the composition, such as 35% to 60% by weight, such as 40% to 50% by weight.

The composition may comprise water in an amount of at least 30% by weight based on total weight of the composition, such as at least 35% by weight, such as at least 40% by weight. The composition may comprise water in an amount of no more than 60% by weight based on total weight of the composition, such as no more than 50% by weight, such as no more than 45% by weight. The composition may comprise water in an amount of 30% to 60% by weight based on total weight of the composition, such as 35% to 50% by weight, such as 40% to 45% by weight.

The carrier may comprise a ratio of phospholipid to water of at least 1:4, such as at least 3:7, such as at least 2:3, such as 1:1. The carrier may comprise a ratio of phospholipid to water of no more than 4:1, such as no more than 7:3, such as no more than 3:2. The carrier may comprise a ratio of phospholipid to water of 1:4 to 4:1, such as 3:7 to 7:3, such as 2:3 to 3:2.

The composition may further comprise, consist essentially of, or consist of an ionophore. As used herein, “ionophore” refers to a compound capable of transporting ions across a lipid membrane. Ionophores that may be used in the compositions disclosed herein include but are not limited to a flavonoid, monensin, lasalocid, salinomycin, narasin, maduramicin, laidlomycin, semduramicin, pyrithione, zincophorin, or combinations thereof. Examples of suitable flavonoids include but are not limited to apigenin (4′,5,7-trihydroxyflavone), luteolin (3′,4′,5,7-tetrahydroxyflavone), tangeritin (4′,5,6,7,8-pentamethoxyflavone), chrysin (5,7-dihyroxyflavone), 6-hydroxyflavone, quercetin (3,3′,4,4′,5,7-pentahydroxyflavone), and combinations thereof.

The ionophore may be present in the composition in an amount of at least 0.005% by weight based on total weight of the composition, such as at least 0.01% by weight, such as at least 0.02% by weight. The ionophore may be present in the composition in an amount of no more than 1% by weight based on total weight of the composition, such as no more than 0.5% by weight, such as no more than 0.1% by weight. The ionophore may be present in the composition in an amount of 0.005% to 1% by weight based on total weight of the composition, such as 0.01% to 0.5% by weight, such as 0.02% to 0.1% by weight.

The composition may optionally further comprise, consist essentially of. or consist of an amino acid. As used herein, “amino acid” refers to a compound comprising a general structure:

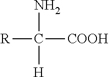

wherein R comprises a hydrogen, a substituted or unsubstituted alkyl group, a substituted or unsubstituted aryl group, a heteroatom, or combinations thereof. Suitable amino acids comprise arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine, tryptophan, selenomethionine, or combinations thereof. The amino acid may comprise the L-enantiomer, the R-enantiomer, or a combination thereof.

The composition may comprise an amino acid in an amount of at least 0.005% by weight based on total weight of the composition, such as at least 0.01% by weight, such as at least 0.015% by weight, such as at least 0.02% by weight, such as at least 0.5% by weight, such as at least 0.75% by weight, such as at least 2% by weight, such as at least 2.5% by weight. The composition may comprise an amino acid in an amount of no more than 10% by weight based on total weight of the composition, such as no more than 7.5% by weight, such as no more than 5% by weight, such as no more than 4% by weight, such as no more than 3.% by weight, such as no more than 1% by weight, such as no more than 0.5% by weight, such as no more than 0.1% by weight, such as no more than 0.05% by weight. The composition may comprise an amino acid in an amount of 0.005% by weight to 10% by weight based on total weight of the composition, such as 0.01% by weight to 7.5% by weight, such as 0.02% by weight to 5% by weight, such as 0.5% by weight to 4% by weight, such as 0.75% by weight to 4% by weight, such as 2% by weight to 4% by weight, such as 2.5% by weight to 3.5% by weight, such as 0.005% by weight to 1% by weight, such as 0.01% by weight to 0.5% by weight, such as 0.015% by weight to 0.1% by weight, such as 0.02% by weight to 0.05% by weight. The composition may comprise a first amino acid, a second amino acid, etc. in varying amounts within the ranges disclosed above, such that the total amount of amino acids is within the ranges disclosed above.

The composition may optionally comprise, consist essentially of, or consist of a peptide. As used herein, “peptide” means a molecule comprising a short chain of amino acids that are linked by peptide bonds. The peptide may comprise an oligopeptide or a polypeptide. As used herein, “oligopeptide” refers to a peptide comprising 2 to 20 amino acid residues. As used herein, “polypeptide” refers to a peptide comprising greater than 20 to less than 50 amino acid residues. Examples of peptides that may be used in the compositions disclosed herein include but are not limited to collagen peptides, antimicrobial peptides, creatine peptides, copper peptides, ipamorelin, follistatin, BPC-157, and combinations thereof.

The composition may comprise a peptide in an amount of at least 1% by weight based on total weight of the composition, such as at least 1.5% by weight, such as at least 2% by weight. The composition may comprise a peptide in an amount of no more than 5% by weight based on total weight of the composition, such as no more than 4% by weight, such as no more than 3.5% by weight. The composition may comprise a peptide in an amount of 1% to 5% by weight based on total weight of the composition, such as 1.5% to 4% by weight, such as 2% to 3.5% by weight.

The composition optionally may further comprise a vitamin or combination of vitamins. As used herein, “vitamin” refers to a molecule essential to normal metabolic functioning that cannot be synthesized naturally by the subject. The vitamin or combination of vitamins may comprise vitamin A, vitamin B₁, vitamin B₂, vitamin B₃, vitamin B₄, vitamin B₅, vitamin B₆, vitamin B₇, vitamin B₈, vitamin B₉, vitamin B₁₀, vitamin B₁₁, vitamin B₁₂, vitamin C, vitamin D, vitamin E, vitamin K, and combinations thereof. Vitamin C may comprise ascorbyl palmitate.

The composition may comprise the vitamin in an amount of at least 0.1% by weight based on total weight of the composition, such as at least 0.5% by weight, such as at least 1% by weight. The composition may comprise the vitamin in an amount of no more than 5% by weight based on total weight of the composition, such as no more than 4% by weight, such as no more than 3% by weight. The composition may comprise the vitamin in an amount of 0.1% to 5% by weight based on total weight of the composition, such as 0.5% to 4% by weight, such as 1% to 3% by weight.

The composition may optionally further comprise a mineral, a mineral source, or combinations thereof. The mineral may comprise selenium, sulfur, iron, chlorine, cobalt, copper, manganese, molybdenum, iodine, zinc, and combinations thereof. The mineral source may comprise any source of the minerals disclosed herein. The composition may comprise zinc and a second mineral in addition to the zinc.

The composition may comprise zinc in an amount of at least 0.01% by weight based on total weight of the composition, such as at least 0.02% by weight, such as at least 0.03% by weight. The composition may comprise zinc in an amount of no more than 0.1% by weight based on total weight of the composition, such as no more than 0.08% by weight, such as no more than 0.06% by weight. The composition may comprise zinc in an amount of 0.01% to 0.1% by weight based on total weight of the composition, such as 0.02% to 0.08% by weight, such as 0.03% to 0.06% by weight.

The composition may comprise a mineral other than or in addition to the zinc source in an amount of at least 0.01% by weight based on total weight of the composition, such as at least 0.03% by weight, such as at least 0.05% by weight, such as at least 0.07% by weight. The composition may comprise a mineral other than or in addition to the zinc source in an amount of no more than 0.5% by weight based on total weight of the composition, such as no more than 0.2% by weight, such as no more than 0.1% by weight, such as no more than 0.08%, such as no more than 0.06%. The composition may comprise a mineral other than or in addition to the zinc source in an amount of 0.01% to 0.5% by weight based on total weight of the composition, such as 0.01% to 0.2% by weight, such as 0.01% to 0.1% by weight, such as 0.01% to 0.08% by weight, such as 0.01% to 0.06% by weight, such as 0.03% to 0.2% by weight, such as 0.03% to 0.08% by weight, such as 0.03% to 0.06% by weight, such as 0.05% to 0.1% by weight, such as 0.05% to 0.08% by weight, such as 0.07% to 0.1% by weight.

Suitable zinc sources include but are not limited to zinc picolinate, zinc citrate, zinc acetate, zinc glycerate, zinc monomethionine, and combinations thereof.

The composition may comprise the zinc source in an amount of at least 0.01% by weight based on total weight of the composition, such as at least 0.03% by weight, such as at least 0.05% by weight, such as at least 0.07% by weight. The composition may comprise the zinc source in an amount of no more than 1% by weight based on total weight of the composition, such as no more than 0.5% by weight, such as no more than 0.2% by weight, such as no more than 0.1% by weight. The composition may comprise the zinc source in an amount of 0.01% to 1% by weight based on total weight of the composition, such as 0.03% to 0.5% by weight, such as 0.05% to 0.2% by weight, such as 0.07% to 0.1% by weight.

The mineral in addition to the zinc may be provided by a second mineral source. The composition may comprise the second mineral source in an amount of at least 0.01% by weight based on total weight of the composition, such as at least 0.03% by weight, such as at least 0.05% by weight, such as at least 0.07% by weight. The composition may comprise the second mineral source in an amount of no more than 1% by weight based on total weight of the composition, such as no more than 0.5% by weight, such as no more than 0.2% by weight, such as no more than 0.1% by weight. The composition may comprise the second mineral source in an amount of 0.01% to 1% by weight based on total weight of the composition, such as 0.03% to 0.5% by weight, such as 0.05% to 0.2% by weight, such as 0.07% to 0.1% by weight.

The composition may optionally further comprise, consist essentially of, or consist of an anti-inflammatory agent. As used herein, “anti-inflammatory agent” excludes cannabinoids. The anti-inflammatory agent may comprise hyaluronic acid, curcumin, methotrexate, tofacitinib, 6-mercaptopurine, azathioprine, sulfasalazine, mesalazine, olsalazine, chlorophane/hydroxychloroquine, penicillamine, aurothiomalate, azathioprine, colchicine, corticosteroids, a beta-2 adrenoreceptor agonist (such as salbutamol, terbutaline, or salmeterol), a xanthine (such as theophylline or aminophylline), cromoglycate, nedocromil, ketotifen, ipratropium, oxitropium, cyclosporin, FK506, rapamycin, mycophenolate mofetil, leflunomide, ibuprofen, naproxen, a corticosteroid, such as prednisolone, a phosphodiesterase inhibitor, an adenosine agonist, an antithrombotic agent, a complement inhibitor, an adrenergic agent, an agent that interferes with signaling by proinflammatory cytokines such as TNF or IL-1 (e.g., an NIK, IKK, p38, or MAP kinase inhibitor), an IL-1 converting enzyme inhibitor, a T-cell signaling inhibitor (e.g. a kinase inhibitor), a metalloproteinase inhibitor, sulfasalazine, a 6-mercaptopurine, an angiotensin converting enzyme inhibitor, a soluble cytokine receptor (e.g., soluble p55 or p75 TNF receptors and the derivatives p75TNFRigG (etancercept) and p55TNFRigG (lenercept), siL-1RI, siL-1RII, siL-6R), an anti-inflammatory cytokine (e.g., IL-4, IL-10, IL-11, IL-13, or TGF), celecoxib, folic acid, hydroxychloroquine sulfate, rofecoxib, etanercept, infliximab, adalimumab, certolizumab, tocilizumab, abatacept, valdecoxib, sulfasalazine, methylprednisolone, meloxicam, methylprednisolone acetate, gold sodium thiomalate, aspirin, triamcinolone, acetonide, propoxyphene napsylate/apap, folate, nabumetone, diclofenac, piroxicam, etodolac, diclofenac sodium, oxaprozin, diclofenac sodium/misoprostrol, anakrina, salsalate, sulindac, cyanocobalamin/fa/pyridoxine, or combinations thereof.

The composition may comprise the anti-inflammatory agent in an amount of at least 0.01% by weight based on total weight of the composition, such as at least 0.05% by weight, such as at least 0.1% by weight. The composition may comprise the anti-inflammatory agent in an amount of no more than 3% by weight based on total weight of the composition, such as no more than 2% by weight, such as no more than 1% by weight. The composition may comprise the anti-inflammatory agent in an amount of 0.01% to 3% by weight based on total weight of the composition, such as 0.05% to 2% by weight, such as 0.1% to 1% by weight.

The composition optionally may further comprise an essential oil. As used herein, “essential oil” refers to a natural oil containing volatile chemical compounds from plants, which may be extracted from, for example, the flowers, seeds, fruits, roots, bark, and/or sap of a plant. Examples of essential oils that may be used in the present disclosure comprise sage oil, coriander oil, thyme oil, pimento berries oil, rose oil, anise oil, balsam oil, bergamot oil, rosewood oil, camphor oil, cardamom oil, cedar oil, cedar leaf oil, chamomile oil, cinnamon oil, sage oil, clary sage oil, clove oil, clove leaf oil, cypress oil, eucalyptus oil, fennel oil, fistree oil, sea fennel oil, frankincense oil, geranium oil, ginger oil, grapefruit oil, jasmine oil, juniper oil, lavender oil, lemon oil, lemongrass oil, lime oil, mandarin oil, marjoram oil, myrrh oil, menthol, neroli oil, orange oil, patchouli oil, pepper oil, black pepper oil, petitgrain oil, pine seed oil, pine needle oil, rosehip oil, rose otto oil, rosemary oil, sandalwood oil, spearmint oil, spikenard oil, vetiver oil, walnut oil, whitepine oil, wintergreen oil, ylang, and combinations thereof.

The composition may comprise the essential oil in an amount of at least 0.001% by weight based on total weight of the composition, such as at least 0.005% by weight, such as at least 0.01% by weight. The composition may comprise the essential oil in an amount of no more than 0.05% by weight based on total weight of the composition, such as no more than 0.04% by weight, such as no more than 0.03% by weight. The composition may comprise the essential oil in an amount of 0.001% to 0.05% by weight based on total weight of the composition, such as 0.005% to 0.04% by weight, such as 0.01% to 0.03% by weight.

The composition may optionally further comprise, consist essentially of, or consist of a brassinosteroid. Brassinosteroids that may be used in the compositions disclosed herein include but are not limited to 24(S) ethylbrassinone analogs, (22R,23R,24S)-2alpha, 3alpha5alpha,22,23-pentahydroxy-stigmastan-6-one, (22R,23R,24S)-3beta-bromo-5alpha,22,23-trihydroxystigmastan-6-one, (22S,23S,24S)-2alpha,3alpha,22,23-tetrahydroxy-5alpha,stigmastan-6-one, (22R,23R,24S)-3beta-acetoxy-22,23-dihydroxy-5alpha-cholestan-6-one, (22S,23S,24S)-3beta-bromo-22,23-dihydroxy-5alpha-cholestan-6-one, (22S,23S,24S)-3beta-bromo-5alpha,22,23-trihydroxy-stigmastan-6-one, and (22S,23S)-3β-bromo-5a,22,23-trihydroxystigmastan-6-one.

The composition may comprise the brassinosteroid in an amount of at least 0.001% by weight based on total weight of the composition, such as at least 0.005% by weight, such as at least 0.01% by weight. The composition may comprise the brassinosteroid in an amount of no more than 0.05% by weight based on total weight of the composition, such as no more than 0.04% by weight, such as no more than 0.03% by weight. The composition may comprise the brassinosteroid in an amount of 0.001% to 0.05% by weight based on total weight of the composition, such as 0.005% to 0.04% by weight, such as 0.01% to 0.03% by weight.

The composition may optionally comprise, consist essentially of, or consist of an antibiotic. Antibiotics that may be used in the compositions disclosed herein include but are not limited to subtilosin, ampicillin, bacampicillin, carbenicillin indanyl, mezlocillin, piperacillin, ticarcillin, amoxicillin-clavulanic acid, ampicillin-sulbactam, benzylpenicillin, cloxacillin, dicloxacillin, methicillin, oxacillin, penicillin G, penicillin V, piperacillin tazobactam, clavulanic acid, nafcillin, procaine penicillin, cefadroxil, cefazolin, cephalexin, cephalothin, cephapirin, cephradine, cefaclor, cefamandole, cefonicid, cefotetan, cefoxitin, cefprozil, cefmetazole, cefuroxime, loracarbef cefdinir, ceftibuten, cefoperazone, cefixime, cefotaxime, cefpodoxime proxetil, cefazidime, ceftizoxime, ceftriaxone, cefepime, azithromycin, clarithromycin, clindamycin, dirithromycin, erythromycin, lincomycin, troleandomycin, cinoxacin, ciprofloxacin, enoxacin, gatifloxacin, grepafloxacin, levoflocaxin, lomefloxacin, moxifloxacin, nalidixic acid, norfloxacin, ofloxacin, sparfloxacin, trovafloxacin, oxolinic acid, gemifloxacin, perfloxacin, imipenem-cilastatin meropenem, axtreonam, or combinations thereof.

The composition may comprise any therapeutically effective amount of the antibiotic. For example, the composition may comprise the antibiotic in an amount of at least 0.001% by weight based on total weight of the composition, such as at least 0.005% by weight, such as at least 0.01% by weight. The composition may comprise the antibiotic in an amount of no more than 0.05% by weight based on total weight of the composition, such as no more than 0.04% by weight, such as no more than 0.03% by weight. The composition may comprise the antibiotic in an amount of 0.001% to 0.05% by weight based on total weight of the composition, such as 0.005% to 0.04% by weight, such as 0.01% to 0.03% by weight.

The composition may optionally further comprise, consist essentially of, or consist of additional components, such as ascorbic acid, ascorbic acid derivatives, glucosamine ascorbate, arginine ascorbate, lysine ascorbate, nictinamide ascorbate, niacin ascorbate, allantoin ascorbate, creatine ascorbate, creatinine ascorbate, chondroitin ascorbate, chitosan ascorbate, DNA ascorbate, carnosine ascorbate, tocotrienol, rutin, hesperidin, diosmin, mangiferin, mangostin, cyanidin, astaxanthin, lutein, lycopene, resveratrol, tetrahydrocurcumin, rasmarinic acid, hypericin, allegic acid, chlorogenic acid, oleuropein, α-lipoic acid, niacinamide, lipoate, glutathione, andrographolide, carnosine, niacinamide, potentilla erecta extract, polyphenols, grape seed extract, pynenogenol, pyridoxine, magnolol, honokiol, paeconol, resacetophenone, quinacetophenone, arbutin, kojic acid, and combinations thereof.

The composition may comprise each of the additional components in an amount of at least 1% by weight based on total weight of the composition, such as at least 1.5% by weight, such as at least 2% by weight. The composition may comprise each of the additional components in an amount of no more than 10% by weight based on total weight of the composition, such as no more than 8% by weight, such as no more than 5% by weight. The composition may comprise each additional component in an amount of 1% to 10% by weight based on total weight of the composition, such as 1.5% to 8% by weight, such as 2% to 5% by weight.

Without wishing to be bound by theory, it is believed that the compositions disclosed herein surprisingly may reduce the presence of reactive oxygen species in a subject's cells. As used herein, “reactive oxygen species” refers to molecules with oxygen that comprise unpaired electrons that can react with other substances. Reactive oxygen species can cause irreversible damage to DNA as they oxidize and modify some cellular components and prevent them from performing their original functions. During times of environmental stress, such as ultraviolet or heat exposure, reactive oxygen species can increase dramatically, which may result in oxidative stress. Oxidative stress may irreversibly affect the development of tissues.

Without wishing to be bound by theory, it is also believed that the compositions disclosed herein surprisingly may activate immune cells and/or repair tissue damage associated with oxidative stress. The compositions disclosed herein may activate CB2 receptors expressed in immune cells. CB2 receptors can modulate immune cell functions and reduce inflammation. By activating these receptors, the compositions disclosed herein may stimulate cellular repair mechanisms connected to immune cells and/or reduce inflammation.

The composition may be formulated as a topical composition. As used herein, “topical composition” refers to a composition that is formulated to be applied to the surface of the skin. The topical composition may optionally comprise exfoliating agents. The exfoliating agents may comprise a chemical exfoliant or exfoliant particles. Examples of suitable exfoliating agents include minerals, organic particles, water-swellable pulverulent polymers (powders or beads), polyethylene particles (beads or powders), jojoba spheres, ground shells of fruit stones, pumice stone, glass beads, aluminum oxide, or combinations thereof. The topical composition may be in the form of a cream, a liniment, a balm, a lotion, a gel, or an ointment.

The topical composition may optionally comprise additional skin care additives. Suitable skin care additives include but are not limited to cleaning agents, skin conditioning agents, perfumes, sunscreen and/or sunblocking compounds, pigments, moisturizers, detergents, thickening agents, emulsifiers, humectants, emollients, deodorant actives, dermatologically acceptable carriers, surfactants, and combinations thereof. The topical composition may further comprise additional components, such as skin soothing agents, healing agents, anti-aging agents, skin moisturizing agents, anti-wrinkle agents, anti-atrophy agents, skin smoothing agents, antimicrobial agents, anti-inflammatory agents, anti-pruriginous agents, external anesthetic agents, antiviral agents, keratolytic agents, free radical scavengers, antiseborrheic agents, depigmenting or propigmenting agents, antiglycation agents, tightening agents stimulating the synthesis of dermal or epidermal macromolecules and/or preventing their degradation, anticellulite agents, slimming agents, agents that may act on microcirculation, agents that may act on the metabolism, cosmetic astrigents, anti-acne agents, anti-caking agents, anti-foaming agents, buffering agents, bulking agents, chelating agents, chemical additives, pH adjusters, pH regulators, sequestrants, skin bleaching and lightening agents, skin tanning agents, skin soothing and/or healing agents and derivatives, skin treatment agents, preservatives, colorants and dyes, or combinations thereof.

The compositions disclosed herein may be formulated as a tincture. As used herein, “tincture” refers to a composition formed by soaking plant parts in a liquid to form a liquid infused with plant extracts. Tinctures provide a method for oral administration of the plant extract. Tinctures are prepared by mixing the plant parts with a suitable solvent wherein a component or components of the plant part are extracted into the solvent. Suitable tincture solvents used in the present disclosure include pharmacologically acceptable solvents such as organic solvents, water-based solvents, alcohols other than the long-chain alcohol, and other orally administrable solvents.

The present disclosure is further directed to a method comprising, consisting essentially of, or consisting of administering to a subject any of the compositions disclosed herein.

The compositions disclosed herein may be administered to treat various disorders, including but not limited to osteoporosis or low bone density; inflammatory disorders such as arthritis; kidney disease; acute kidney trauma; herpes simplex virus 1 and 2; shingles; hypertension; viral infections such as viral pneumonia; bacterial infections such as bacterial pneumonia and S. pyrogenes infections; high cholesterol; high triglyceride levels; and metabolic disease. Administration of the compositions disclosed herein may also result in improved immune functioning, enhanced metabolism, and accelerated healing time of, for example, cuts, bruises, broken bones, tendon and ligament damage, sprains and strains, and the like. Administration of the compositions disclosed herein may also result in anti-aging effects, including but not limited to the appearance of fine lines and wrinkles, liver function improvement, and blood vessel flexibility improvement. The following table provides exemplary conditions that may be treated by administration of the compositions disclosed herein, suggested dosage for treatment thereof, and anticipated outcome.

| Pathology | Usage/Duration | Anticipated Outcome |
|---|---|---|
| Obesity | 5 drops three times per day | Measurable decrease in body |
| | administered sublingually 10 | mass |
| | minutes before meals for 120- | |
| | 180 days | |
| Osteoporosis/Bone | 5 drops two times per day | Increased bone density |
| Density | administered sublingually for | |
| | 60-180 | |
| | Remove all pharmaceutical | |
| | medications | |
| Arthritis | 5 drops two times per day | Increased bone density |
| | administered sublingually for | reduction; osteoclasts and |
| | 14-60 days | alkaline phosphatase resulting |
| | | in mild to moderate pain relief. |
| Chronic Kidney | 5 drops three times per day | Increased glomerular filtration |
| Disease or Acute | administered sublingually for | rate (GFR) >15%, Decreased |
| Kidney Trauma | 30-60 days | creatinine >15% |
| Blisters | 5 drops two times per day | Full prevention or full |
| a) Herpes Simplex 1 | administered sublingually for | resolution 1-10 days following |
| and 2 | maintenance/prevention | initiation of treatment. |
| b) Herpes Zoster | 5 drops three times per day | |
| (Shingles) | administered sublingually and | |
| | topical administration for | |
| | acute breakouts until blisters | |
| | are healed | |
| Hypertension | 5 drops three times per day | Removal of hypertension |
| | administered sublingually for | medications. |
| | 90-180 days. | Removal of water pills or |
| | | hydrochlorothiazide with |
| | | additional CBD and/or weight |
| | | loss. |
| Anti-Viral Anything | 5 drops four times per day | Complete resolution of |
| including viral | administered sublingually up | infection. |
| pneumonia/decreased | (may be administered up to six | |
| O2 stats | times daily). | |
| Anti-Bacterial | 5 drops four times a day | Complete resolution of |
| (Indirect effect) | administered sublingually. | infection. |
| including bacterial | Can use more depending on | |
| pneumonia, S. | severity and age. | |
| pyrogenes, etc. | | |

The composition may be administered in any suitable manner, including but not limited to orally or topically. Oral administration may comprise sublingual administration.

At least 1 drop of a tincture comprising one of the compositions disclosed herein may be administered, such as at least 3 drops, such as at least 5 drops. No more than 20 drops of a tincture comprising one of the compositions disclosed herein may be administered, such as no more than 15 drops, such as no more than 10 drops. 1 to 20 drops of a tincture comprising one of the compositions disclosed herein may be administered, such as 3 to 15 drops, such as 5 to 10 drops.

When administered orally, such as sublingually, the composition may be administered in a dose of at least 50 μL, such as 50 μL to 1.0 mL, such as 50 μL to 0.75 mL, such as 50 μL to 0.5 mL, such as 50 μL to 0.25 mL, such as 50 μL to 200 μL, such as 50 μL to 150 VL. The composition may be administered at a dose of 2 mg of the composition per 1 kg of the subject's weight to 500 mg of the composition per 1 kg of the subject's weight, such as 2 mg/kg to 350 mg/kg, such as 2 mg/kg to 200 mg/kg, such as 2 mg/kg to 100 mg/kg, such as 2 mg/kg to 50 mg/kg.

The method described herein may be repeated 1, 2, 3, 4, or more times per day, resulting in a daily administration of a total daily dose of 50 μL to 4.0 mL per day. Single or multiple administrations per day can be carried out each day or 2, 3, 4, or 5 times per week for 1 month or more, 2 months or more, 3 months or more, 1 year or more, or throughout the lifetime of the patient.

The composition may alternatively be administered orally, such as by a tablet or capsule comprising the composition. The tablet or capsule may comprise a dosage of the composition of at least 20 mg, such as at least 30 mg. such as at least 40 mg. The tablet or capsule may comprise a dosage of the composition of no more than 200 mg, such as no more than 150 mg, such as no more than 100 mg. The tablet or capsule may comprise a dosage of the composition of 20 mg to 200 mg, such as 30 mg to 150 mg, such as 40 mg to 100 mg.

Illustrating the disclosed subject matter are the following examples that are not to be considered as limiting the disclosure to their details. All parts and percentages in the examples, as well as throughout the specification, are by weight unless otherwise indicated.

### Examples

A male patient presented with low albumin levels, high creatinine levels and a high albumin/creatinine ratio. The patient sublingually self-administered daily ten to twenty drops (approximately 1 mL) of a tincture comprising the composition described in Table 1 for 96 days. The patient's albumin and creatinine levels were tested by urinalysis prior to initiating treatment and on day 96 of treatment. The results are provided in Table 2.

TABLE 1

| Component | Dosage¹ | |
|---|---|---|
| Cannabidiol | 4.2 | mg |
| Quercetin | 333 | μg |
| Glutathione | 8.3 | mg |
| Lysine | 8.3 | mg |
| Policosanol | 4.0 | mg |
| Selenomethionine | 333 | μg |
| Ascorbyl palmitate | 4.2 | mg |
| Zinc citrate | 1 | mg |
| Olive oil | 14 | g |
| Deionized water | 10 | g |
| Sunflower lecithin | 1.5 | g |

¹Dosages are per 0.25 mL of composition.

TABLE 2

| | |
|---|---|
| Day 0 | Day 96 |

| | | | | |
|---|---|---|---|---|
| Creatinine | 104 | mg/dL | 184 | mg/dL |
| Albumin | 311 | mg/L | 1,843 | mg/L |

| | | |
|---|---|---|
| Albumin/Creatinine Ratio | 299 mg/1 g | 1,002 mg/1 g |

Whereas aspects of the disclosure have been described in detail, it will be appreciated by those skilled in the art that various modifications and alternatives to those details could be developed in light of the overall teachings of the disclosure. Accordingly, the particular arrangements disclosed are meant to be illustrative only and not limiting as to the scope of the disclosure which is to be given the full breadth of the claims appended and any and all equivalents thereof.

We claim:

1. A composition comprising:
a long chain alcohol comprising octacosanol, hexacosanol, and/or triacontanol in an amount of at least 50% by weight based on total weight of the long chain alcohol;
a cannabinoid; and
a carrier.

2. The composition of claim 1, wherein the composition comprises:
the long chain alcohol in an amount of 0.01% to 5% by weight based on total weight of the composition;
the cannabinoid in an amount of 0.1% to 5% by weight based on total weight of the composition; and/or
the carrier in an amount of 50% to 95% by weight, based on total weight of the composition.

3. The composition of claim 1, wherein the long chain alcohol comprises a ratio of hexacosanol to octacosanol of 1:19 to 2:3: a ratio of hexacosanol to triacontanol of 1:4 to 4:1; and/or a ratio of triacontanol to octacosanol of 1:19 to 2:3.

4-9. (canceled)

10. The composition of claim 1, wherein the cannabinoid comprises cannabidiol.

11. The composition of claim 10, wherein the composition comprises the cannabidiol in an amount of over 50% by weight based on total weight of the cannabinoid.

12-17. (canceled)

18. The composition of claim 1, further comprising an ionophore; glutathione; an amino acid; a vitamin; a mineral, mineral source, or a combination thereof; an anti-inflammatory agent; and/or an essential oil.

19. The composition of claim 18, wherein the composition comprises;
the ionophore in an amount of 0.005% to 1% by weight based on total weight of the composition,
glutathione in an amount of 1% by weight to 10% by weight based on total weight of the composition;
the amino acid in an amount of 1% to 5% by weight based on total weight of the composition;
the vitamin in an amount of 0.1% to 5% by weight based on total weight of the composition;
the mineral in an amount of 0.01% to 0.5% by weight based on total weight of the composition;
the anti-inflammatory agent in an amount of 0.01% to 3% by weight based on total weight of the composition; and/or
the essential oil in an amount of 0.01% to 5% by weight based on total weight of the composition.

20. The composition of claim 18, wherein:
the ionophore comprises a flavonoid;
the amino acid comprises lysine, selenomethionine, or a combination thereof;
the vitamin comprises an A vitamin, a B vitamin, a C vitamin, a D vitamin, an E vitamin, a K vitamin, or a combination thereof; and/or
the mineral comprises selenium, sulfur, iron, cobalt, copper, manganese, molybdenum, iodine, zinc, or a combination thereof.

21-31. (canceled)

32. The composition of claim 20, wherein the composition comprises zinc in an amount of 0.01% to 0.1% by weight based on total weight of the composition.

33-39. (canceled)

40. A composition comprising:
a long chain alcohol; a cannabidiol; a vitamin; an ionophore; an amino acid; zinc;
glutathione; and a carrier.

41. The composition of claim 40 comprising:
the long chain alcohol in an amount of 0.01% to 5% by weight based on total weight of the composition;
the cannabidiol in an amount of 0.1% to 5% by weight based on total weight of the composition;
the vitamin in an amount of 0.1% to 5% by weight based on total weight of the composition;
the ionophore in an amount of 0.005% to 1% by weight based on total weight of the composition;
the amino acid in an amount of 1% to 5% by weight based on total weight of the composition;
the zinc in an amount of 0.01% to 0.1% by weight based on total weight of the composition;
the glutathione in an amount of 1% to 10% by weight based on total weight of the composition; and/or
the carrier in an amount of 50% to 95% by weight based on total weight of the composition.

42. The composition of claim 1, wherein the composition comprises tetrahydrocannabinol in an amount of less than 3% by weight based on total weight of the composition.

43. A topical composition comprising the composition of claim 1.

44. (canceled)

45. A tincture comprising the composition of claim 1.

46. A method comprising administering to a subject the composition of claim 1.

47. (canceled)

48. The method of claim 46, wherein the composition is administered orally or topically.

49. The method of claim 48, wherein the oral administration comprises sublingual administration.

50-51. (canceled)

52. The method of claim 46, wherein the composition is administered at a total daily dose of 50 μL to 4.0 mL.

53. The method of claim 46, wherein the composition is administered at a dose of 2 mg of the composition per 1 kg of the subject's weight to 500 mg of the composition per 1 kg of the subject's weight.

54-63. (canceled)

64. The composition of claim 40, wherein the composition comprises tetrahydrocannabinol in an amount of less than 3% by weight based on total weight of the composition.
